(19) United States
(12) Patent Application Publication
(10) Pub. No.: US 2022/0336109 A1
(43) Pub. Date: Oct. 20, 2022

(54) APPLICATION FOR TRACKING PROGRESSION AND ISOLATING CAUSES OF ADVERSE MEDICAL CONDITIONS

(71) Applicant: OptimDosing, LLC, Farmington Hills, MI (US)

(72) Inventors: Kenneth I. KOHN, West Bloomfield, MI (US); David INWALD, Berkley, MI (US); Caitlin Joline BROWN, Ashburn, VA (US)

(73) Assignee: OptimDosing, LLC, Farmington Hills, MI (US)

(21) Appl. No.: 17/635,982

(22) PCT Filed: Aug. 14, 2020
(86) PCT No.: PCT/US20/46399

§ 371 Date: Feb. 16, 2022

Related U.S. Application Data
(60) Provisional application No. 62/887,940, filed on Aug. 16, 2019.
(60) Provisional application No. 62/930,851, filed on Nov. 5, 2019.
(60) Provisional application No. 62/931,314, filed on Nov. 6, 2019.
(60) Provisional application No. 62/988,974, filed on Mar. 13, 2020.
(60) Provisional application No. 63/023,806, filed on May 12, 2020.
(60) Provisional application No. 63/035,574, filed on Jun. 5, 2020.

(51) Int. Cl. G16H 50/80; G16H 20/30; G16H 20/70; G16H 20/60; G16H 10/20; G16H 10/60; A61B 5/00

(52) U.S. Cl. CPC G16H 50/80; G16H 20/30; G16H 20/70; G16H 20/60; G16H 10/20; G16H 10/60; A61B 5/4824; A61B 5/7275

(57) ABSTRACT

An application for tracking disease, pain, and mental health symptom triggers including an input module for inputting variables from a user in electronic communication with an output variable module, an analysis module for analyzing input variables and output variables, and an output module for presenting results to the user. A method of tracking disease, pain, and mental health triggers, by a user inputting data about nutrition, medication, lifestyle, symptoms, pain, and user defined metrics in an application, performing an analysis on the data, and outputting a result from the data identifying daily activities that effect the user's disease/mental health and trigger symptoms. A method of preventing adverse events.

### BACKGROUND OF THE INVENTION

### 1. Technical Field

The present invention relates to methods of tracking daily activity and symptoms of diseases and mental health issues. More specifically, the present invention relates to methods of tracking symptoms of adverse events to predict adverse events related to various diseases, such as digestive diseases, migraine, panic attacks, pain, contagious diseases, and others.

### 2. Background Art

Nearly one-fifth of the American public faces chronic digestive problems. This includes 25 to 45 million people with Irritable Bowel Syndrome (IBS) and an additional 3 million with the more serious and potentially life threatening Inflammatory Bowel Disease (IBD, which includes Crohn's Disease and Ulcerative Colitis). IBS can be caused by muscle contractions in the intestine, abnormalities in the nervous system, inflammation in the intestines, severe infections, or changes in the gut microbiome. Symptoms of IBS include cramping, abdominal pain, bloating, gas, diarrhea, constipation, and combinations thereof. While severe symptoms can be treated with medicine, many people can relieve their symptoms by managing their diet and lifestyle. IBD, on the other hand, is a chronic inflammation of the digestive tract and can involve the symptoms of diarrhea, fever and fatigue, abdominal pain and cramping, blood in the stool, reduced appetite, and weight loss. IBD may be caused by immune system malfunctions and can be aggravated by diet and lifestyle.

Several platforms exist that help sufferers of digestive diseases track their symptoms. Oshi is an IBD-focused platform that includes tracking as one facet. Dimensions captured include disease activity, stress, physical activity, sleep, and diet adherence. The diet adherence requires the individual to tell it what foods they need to avoid, then it helps you track if they have avoided them. The symptoms trackers tallies bms, pain, bleeding (on a scale of 0 to 3 OR 4-10), and how well the individual feels the disease is in control. The insights possible with this platform are therefore very limited.

FlareDown tracks disease symptoms and medication. However, the Crohn's default daily check-in is just “How was your condition today?” and responding from “not active” to “extremely active”. Individuals can add other symptoms, but the list is vast and not well-organized or user friendly. Individuals also log the medications they took, as well as the foods they ate. However, the foods are “search for whatever food you want to track” approach, which is neither user friendly, not helpful. Reminders to check in are limited to email.

SymptomTracker allows users to track symptoms over time (from pain to motivation). However, it does not include any other information and so cannot speak to what may be causing the changes in the tracked symptom.

There remains a need for chronic sufferers of digestive diseases to identify how their day-to-day lives impact their symptoms.

It would also be useful to track symptoms and triggers of various other diseases and mental health problems, so that individuals can learn how to avoid or change situations that trigger these issues as well as predict when an adverse event will occur to prevent the adverse event.

For example, migraines are a type of headache that recur with moderate to severe pain, and can include nausea, weakness, and sensitivities to light and sound in about 12 percent of individuals in the United States. They are thought to be genetic. Many different factors can trigger migraines, such as stress, anxiety, hormonal changes, bright or flashing lights, loud noises, strong smells, medicines, sleep patterns, sudden weather changes, overexertion, tobacco use, caffeine or lack of caffeine, missed meals, and foods and additives. Treatments focus on relieving symptoms and preventing further attacks and include medicines such as pain relievers, calcitonin gene-related peptide injections, BOTOX® injections, mild anesthesia treatments, stress management, rest, and hormone therapy. Doctors suggest tracking triggers to avoid them. Apps exist to help individuals with this. Migraine Buddy allows an individual to record migraine frequency and duration, pain location and intensity, symptoms and medications, and can help identify triggers. Ouchie allows an individual to post data about where they feel pain, pain intensity, and treatments, and this is shared with an app community with similar symptoms where they can share tips for reducing pain.

The concept of measuring patient pain is a challenging topic which is highly relevant in a time where opioid concern and addiction is a huge issue around the world. Patients are asked to rate pain on a scale of one to ten, which is subjective and up to the patient to determine. With no ability to compare pain levels to a known level, the patients provide a number that can mean they are in extreme pain when in fact, they are not in sufficient pain to require pain medication. On the other hand, patients in pain can provide a number lower than their actual pain level resulting in incorrect treatment of pain and lack of pain medication. This can lead to patients misstating pain levels to insure pain medication.

For the doctor, this leaves a patient's pain condition up to judgment and requires balancing the patient provided pain number with personal experience. While this would normally work, opioid addiction due to overprescribing and bad faith pharmaceutical distribution has led to litigation and legal ramifications for doctors that want to provide proper dosing. In certain cases, the pendulum has swung so far as to have doctors avoid prescribing pain medication needed by the patients to avoid any legal risks.

While addiction and overprescribing are serious issues, patients that truly need pain relief are left in an untenable position of providing pain numbers that are subjective and doctors that cannot rely on what the patient is saying. However, not treating pain is dangerous for the patient and simply cannot be ignored. In an article written by Forest Tennant, MD, DrPH, in Practical Pain Management, Volume 10, Issue 8,

“Although severe pain can have profound and negative impacts on the cardiovascular (CV) system, this complication has received scant attention. Pain may affect the CV system by multiple mechanisms, and sudden CV death may occur in chronic pain patients who experience a severe pain flare. One of the goals of pain treatment should be to stabilize and bring homeostasis to a pain patient's CV system. This is particularly the case with older patients who have either overt or covert cardiovascular disease or who may be at risk of developing it.”

Thus, not treating pain can be deadly for the patient. Therefore, there is a need for something better that can correlate pain to a pain scale and provide factual data for the doctor to verify pain and record the data in a patient chart for documentation.

Dr. Tennant also writes:

“Pain causes elevation of blood pressure and pulse rate by two basic mechanisms that may simultaneously operate. The sympathetic (autonomic) nervous system is stimulated by electrical pain signals that reach the central nervous system. This may occur in acute pain, during flares, or breakthrough pain. The aberrant, neuronatomic brain changes that may occur with severe constant pain appears to be capable of producing continuous sympathetic discharge. Pain also signals the hypothalamus and pituitary to release adrenocorticotropin hormone (ACTH) which stimulates the adrenal glands to release adrenalin with subsequent elevation of pulse and blood pressure. Recognition of sympathetic stimulation is a useful clinical tool to help guide therapy and diagnose uncontrolled pain. Besides hypertension and tachycardia, sympathetic discharge also produces mydriasis (dilated pupil), diaphoresis (sweating), hyperactive reflexes, nausea, diarrhea, vasoconstriction (cold hands and feet), anorexia, and insomnia.”

Therefore, there remains a need for a method of accurately tracking pain and evaluating pain in patients.

Panic or anxiety attacks cause intense fear in an individual without a real danger or cause and begin suddenly. Symptoms include a sense of impending doom or danger, fear of loss of control or death, rapid and pounding heart rate, sweating, trembling or shaking, shortness of breath, chills, hot flashes, nausea, abdominal cramping, chest pain, headaches, dizziness, lightheadedness, faintness, numbness or tingling sensation, or feeling of unreality or detachment. Causes can include genetics, stress, or changes in brain function. It is also suggested to track triggers and symptoms to cope with anxiety attacks. For example, Anxiety Reliever is an app that allows an individual to track symptoms and provides relaxation exercises.

Apps also exist to help individuals having suicidal thoughts. MY3 allows a user to define a network of individuals with whom they can reach out to when suicidal thoughts occur. Suicidal Safety Plan designs a plan for individuals to follow to cope with suicidal thoughts.

Infectious diseases are diseases caused by pathogenic microorganisms such as bacteria, viruses, parasites, or fungi that can be spread from person to person, either directly or indirectly.

Coronavirus Disease 2019 (COVID-19) is a severe acute respiratory syndrome (SARS) coronavirus 2 that originated in 2019 in Wuhan, China, and has quickly spread around the world. The viral infection is spread from person to person by respiratory droplets. Symptoms include fever, cough, and shortness of breath and it is very similar to influenza. While tests are available to identify COVID-19, they are generally not being used on people with milder symptoms due to the lack of number of tests. Individuals who have been identified as having the virus need to quarantine themselves. It would be advantageous to track individual's symptoms if they are not feeling well as well as tracking habits of quarantine individuals to ensure compliance.

Therefore, there remains a need for a method of tracking many different diseases and mental health issues wherein the identification of triggers for adverse events is a complex process not easily identifiable by a practitioner merely reviewing a patient's daily log, and a need for predicting adverse events so that they can be avoided or treated in time.

### SUMMARY OF THE INVENTION

The present invention provides for an application for tracking disease, pain, and mental health symptom triggers stored on non-transitory computer readable media including an input module for inputting variables from a user in electronic communication with an output variable module, an analysis module for analyzing input variables and output variables, and an output module for presenting results to the user.

The present invention provides for a method of tracking disease, pain, and mental health triggers, by a user inputting data about nutrition, medication, lifestyle, symptoms, pain, and user defined metrics in an application stored on non-transitory computer readable media, performing an analysis on the data, and outputting a result from the data identifying daily activities that effect the user's disease/mental health and trigger symptoms.

The present invention also provides for a method of preventing adverse events, by a user inputting data about nutrition, medication, lifestyle, symptoms, pain and user defined metrics in an application stored on non-transitory computer readable media, integrating data from outside devices and outside databases, performing an analysis on the data, and outputting a result from the data identifying that an adverse event is likely to occur at a later time point.

### DESCRIPTION OF THE DRAWINGS

Other advantages of the present invention are readily appreciated as the same becomes better understood by reference to the following detailed description when considered in connection with the accompanying drawings wherein:

FIG. 1 is a diagram of the flow of information in the application and method; and

FIG. 2 is a macro-level systems design of the present invention.

### DETAILED DESCRIPTION OF THE INVENTION

The present invention generally provides for a user friendly application (shown at 10 in the FIGURES) and method of use that quickly captures daily activities, intake, and symptoms of users with diseases and mental health issues to find otherwise hidden patterns in order to determine symptom triggers and effects on their body. The information can be input by the user answering preset questions. Additionally, the information can be input from existing and newly developed outside monitoring devices. These monitoring devices can measure cardiac, circulatory or other physical properties of the user over time. The information gathered is analyzed over time along with patient gathered data gathered over time. This information enables users to tweak their lifestyle and feel better. The information can also be used to predict an adverse event happening at a later time point so that the user can either prevent the adverse event from happening with lifestyle changes or receive treatment to prevent the adverse event.

The term “application” as used herein refers to a computer software application, otherwise known as an “app”, that is run and operated on a mobile device, such as, but not limited to, smart phones (IPHONE® (Apple, Inc.), ANDROID™ devices (Google, Inc.), WINDOWS® devices (Microsoft)), mp3 players (IPOD TOUCH® (Apple, Inc.)), or tablet computers (IPAD® (Apple, Inc.)), especially ones utilizing a touch screen. The application can also be web based and run on a computer or laptop. The application 10 includes any necessary user interface or display and storage components to display the application and store the algorithm running it.

“Diseases and mental health issues” as used herein can include diseases such as digestive disorders or migraines, and mental health issues such as anxiety attacks, or suicidal thoughts, among others. The diseases and mental health issues are preferably ones that are affected by outside triggers such as diet and lifestyle or environment.

“Pain” as used herein can refer to any unpleasant sensation in the body, ranging from mild to severe, as felt through the nervous system. Pain can be localized or systemic. Pain can be acute (lasting less than 30 days), subacute (lasting 1-6 months), or chronic (lasting more than 6 months). Pain can be caused by injury, surgery (especially such as orthopedic device surgery involving knees, hips, shoulders, elbows), cancer, fibromyalgia, arthritis, or peripheral neuropathy.

“Infectious disease” as used herein can include an viral, protozoan, or bacterial disease such as most preferably influenza, measles, or COVID-19, or any of AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, or zika virus.

“Trigger” as used herein, refers to an event or situation that causes or provokes a disease or condition to happen.

“Adverse event” as used herein, refers to any medical occurrence that is undesired in a user. Examples can include, but are not limited to, headaches, nausea, heart attacks, seizures, allergic reactions, hemorrhages, tissue damage, or any other damage to the body. Adverse events can cause disability, permanent damage, or even death.

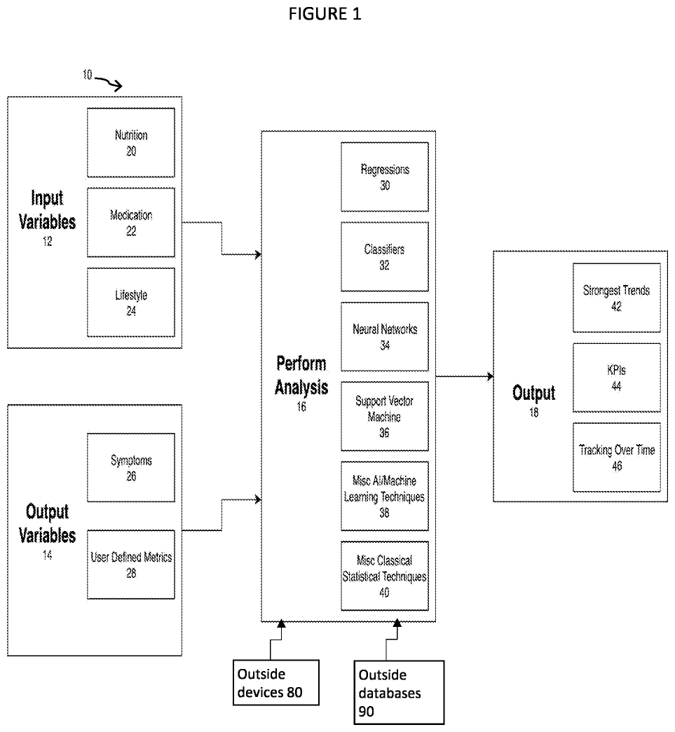

As generally shown in FIG. 1, the application 10 includes an input module 12 for inputting variables from a user in electronic communication with an output variable module 14, an analysis module 16 that analyzes data from the input variables and output variables, and an output module 18 for presenting results to the user. Each of these modules can be run by algorithms stored on non-transitory computer readable media.

The input module 12 can be used to keep a daily log of users' lifestyle and symptoms. The questions are kept very simple so that a user can complete them in 1-2 minutes. The input module 12 can include a nutrition question module 20, a medication question module 22, and a lifestyle question module 24. Questions presented can be answered on a continuous or nominal scale. Input can also be gathered from various medical devices, such as portable monitoring systems, further described below. Accordingly, cardio, vascular, and neuro information can be input.

With the nutrition question module 20, questions can be presented to the user such as (with available answer choices in brackets):

How many servings of grains did you eat today? [0 to 10]

How many servings of fruit did you eat today? [0 to 5 or more]

How many servings of vegetables did you eat today? [0 to 5 or more]

How many servings of dairy did you eat today?

How much sugar did you have today? [0 to 5 scale, way less than average to way more than average]

With the medication question module 22, the user can input any medication they are taking, including vitamins and supplements, with dosing schedules and amounts.

With the lifestyle question module 24, questions can be presented to the user such as (with available answer choices in brackets):

How many hours of sleep did you get last night? [0 to 12+, on 0.5 intervals]

Did you work out today? [yes or no]

Did you take time to relax today? [yes or no]

How stressed did you feel today? [0 to 5 scale]

The output variable module 14 can include a symptom question module 26 and a user defined metrics question module 28.

With the symptom question module 26, questions can be presented to the user such as (with available answer choices in brackets):

How much pain were you in today? [0 to 5 scale]

How many bowel movements did you have today? [0 to 10+, or on Bristol scale]

How many times did you pass blood? [0 to 10+]

Did you have a headache today? [yes or no]

The symptom question module 26 can further include questions related to infectious diseases, such as:

Do you have a cough?

[No]

[Yes→Is it a dry cough or wet cough? (a wet or productive cough means there is fluid in your airways, a dry cough means there is no fluid in your airways), select from wet cough, dry cough, or not sure]

Do you have shortness of breath? [yes or no]

What is your temperature? [Enter #]

[Not sure]→Do you think you have a fever? [yes or no]

Have you had any digestive issues? (e.g., diarrhea, vomiting, etc.)

[No]

[Yes]→

Have you had diarrhea? [yes or no]

Have you felt nauseous? [yes or no]

Have you vomited? [yes or no]

Have you had other abdominal discomfort? [yes or no]

Are you experiencing any of the following? [body aches, chills, fatigue, headache, postnasal drip, runny nose, sinus congestion, skin rash, sneezing, sore throat, swollen glands, watery eyes, loss of smell, loss of taste]

Have you been in contact with anyone with a positive COVID-19 diagnosis?

[No]

[Yes]→When were you in contact?

Where were you in contact?

On a scale of 1 (not at all anxious) to 10 (extremely anxious), how anxious did you feel today?

Have you limited your daily activities?

[No]

[Yes]→Have you self-quarantined? [Yes or No]

With the user defined metrics question module 28, the user can design any other relevant questions and answers that could relate to their disease or condition that can be added to the application 10 to include in an analysis, such as alcohol intake or traveling.

All the data collected from the input module 12 and the output variable module 14 is sent to the analysis module 16. The analysis module 16 can include regressions 30, classifiers 32, neural networks 34, support vector machine 36, miscellaneous AI/machine learning techniques 38, and/or miscellaneous classical statistical techniques 40 in performing the analysis of the data.

In general, the analysis module 16 uses the data to find patterns between how users live and how they feel. By estimating multiple regressions 30 on time lagged variables, the application 10 can find patterns most people cannot casually notice or even calculate if they are keeping careful food diaries. With just one week of data, connections can be identified between how users live and how they feel.

The symptom variables can be used as the dependent variable in a series of regressions 30. The symptom variables include both same day, as entered values and time lagged, such that the first row of data is deleted out to four days later. The nutrition, medication, and lifestyle data measured are used as the independent, or predictor, variables. Linear regressions 30 are then estimated to determine which independent variables cause an increase in the symptoms, or dependent variables. The specific mechanisms are as follows. Users input their symptoms, food intake at a high level, medication intake, and simple lifestyle measures, each on a continuous or nominal (from Likert-type items) scales. The symptom variables include both same day, as entered values, and time lagged, such that the first row of data is deleted out to four days later. The food intake, medication, and lifestyle measured are used as the independent, or predictor, variables. Linear regressions are then estimated to determine which independent variables cause an increase in the symptoms, or dependent variables. Specifically, the symptom variables are then used as the dependent variables in a series of linear, ordinary least regressions. Within the first month of use, three regressions are estimated for each symptom. One regression tests the food variables as the independent variables, one the lifestyle variables, and one the medication variables. Each regression coefficient with alpha<0.2 is flagged to users as a potential factor contributing to their symptoms. After users have inputted a full month of data, one master regression is estimated for each symptom outcome, combining the food, lifestyle, and medication predictor variables, thereby allowing the relative impact across categories to be determined. With the full month of data, the significance level drops to alpha<0.4. This means that the null hypothesis that the relationship between a given factor and the symptom outcome can be rejected with 60 percent certainty. As more data are collected, the threshold for significance will increase as power increases. This will be determined with a series of power analyses. A power analysis looks at the relationship between sample size, in this case the number of days of data collected, significance level, and population effect size, that is the known relationship between factor and outcome, if known. A priori power analyses determine appropriate sample size to achieve adequate power and, in the case of this application, determine the change in significance level needed as the amount of data increases.

Linear regressions 30 test the null hypothesis that the relationship between the independent variable(s) and dependent variable is 0. Unlike traditional data analysis, which requires a 5% alpha level to claim significance, the threshold for flagging potential lifestyle problems is lower. Specifically, the 5% standard level translates to a 95% likelihood that an effect is not due to chance, thereby rejecting the null hypothesis that the relationship is 0. But those who live with chronic illness want to know if there is a good chance, i.e., more than 60%, that a lifestyle choice, food, is causing symptoms. Further, the system can time lag outcome variables to capture the impact of day-to-day life on symptoms the same day, the next day, and the day after that. These regressions 30 serve as the steps in an algorithm.

While regressions 30 can be preferred, other methods of analysis can be used. Classifiers 32 are a broad use of artificial intelligence and machine learning that determine the relationship between input variables and output variables are categories. In the case of the present invention, it can be classified whether or not a specific user's data classifies as fitting the profile of effective lifestyle changes to help improve symptoms.

Time series is a system of data points organized by time. Time then becomes one of the key predictors of an outcome, by looking at autocorrelation, seasonality, and stationarity. Time series enable an understanding of how data vary over time and how changes in a given variable over time compare to changes in other variable over time. Medical conditions inherently change over time, with symptoms becoming better or worse but rarely static. Likewise, nutrition, medication, lifestyle, symptoms, pain, and user defined metrics vary over time. Understanding how adverse symptom outputs change over time as well as how they change over time in conjunction with nutrition, medication, lifestyle, symptoms, pain, and user defined metrics is crucial.

Time series may follow several broad patterns: trends occur when there is an overtime increase or decrease in a data series; seasonal patterns occur when data over time are impacted by external changes at a fixed and known frequency, like time of the week, month, year, etc., and cycles occur when changes in data over time correlate with other, non-fixed external changes. In this application, trends may occur as medical prognosis generally improves or deteriorates. Seasonal patterns may be due to environmental factors that map to seasons or lifestyle choices that vary by day of the week. Cycles would capture changes due to weather and other external factors. Time series analysis will enable the application to account for changes in adverse symptom outcomes over time, as well as changes in adverse symptom outcomes as they relate to related seasonal and cyclical changes in nutrition, medication, lifestyle, symptoms, pain, and user defined metrics.

Neural Networks (NNs) 34 are another broad AI/machine learning technique that can be used to detect patterns in data. Previous use cases for neural networks include real-time translation, facial recognition, and music composition. Neural networks map inputs to outputs via a series of algorithms designed to loosely model the human brain. Specifically, each input is entered as a vector that makes up the left-side layer of a broader neural network. For this application, the inputs include nutrition, medication, lifestyle, symptoms, pain, and user defined metrics The right-side layer of a neural network is the output. In this application, the output includes all adverse symptom outcomes. Between the input and output layers is a hidden layer, which is a weighted sum of the values in the input layer that projects the outcome layer, thereby determining how the inputs work together to create the outputs. This hidden layer would determine how nutrition, medication, lifestyle, symptoms, pain, and user defined metrics work together to create symptom outputs.

Neural networks follow and iterative process between forward and backward propagation. In forward propagation, the weights in factors of the hidden layers are calculated to determine output layer prediction and error probability of that prediction. Backward propagation runs in the opposite direction, bringing higher error likelihood from the right output layer back into the hidden layers to adjust the weights. This in turn decreases the likelihood of error at the output layer. In this application, the hidden layers determine the weights for the different nutrition, medication, lifestyle, symptoms, pain, and user defined metrics to predict adverse symptom outcomes in the output layer. If the error of that prediction exceeds a certain level, back propagation returns to the hidden layers to adjust the weights and increase the probability that the adverse symptom prediction is accurate. Forward and backward propagation are iterative until the output, or adverse symptom event, is predicted with greater certainty.

Deep neural networks add additional hidden layers that aggregate and recombine data from the previous layer. The current application will use the additional layers of deep neural networks to cluster nutrition, medication, lifestyle, symptoms, pain, and user defined metrics together over time. Thus, clusters of behavior across time will more accurately predict adverse symptom outcomes. Deep learning networks use automatic feature extraction, enabling the machine to identify patterns without the need for human intervention, thereby mitigating bias. For the present invention, neural networks are one of the strategies used to identify trends in the data. NN models can be used for analyzing certain symptoms or broadly over the data set.

Support Vector Machines (SVMs) 36 can be used as part of the classification technique to identify certain features. SVMs are supervised learning models that rely on attempting regressions to evaluate which have the strongest fit with the data set.

SVM assumes a binary outcome. In the case of this application: did the adverse symptom occur on a given day or not. SVM then makes a non-probabilistic binary linear classifier by plotting points in space. These points represent factors contributing to the likelihood of the outcome, i.e., nutrition, medication, lifestyle, symptoms, pain, and user defined metrics. The bigger the gap between the clusters, the better the predictive power, as the potential binary outcomes sit relatively farther apart.

In most real world examples, however, the gap between one outcome vs. the other is non-existent, with much overlap. This is likely the case with predicting adverse symptoms, as the predicting nutrition, medication, lifestyle, symptoms, pain, and user defined metrics likely bleed together. To account for this, the application will use the Kernel Trick. Kernal functions compute the similarity between inputs according this formula, where x and y are input vectors, ϕ is a transformation function, and < > refers to the dot product:

K(x,y)<ϕ(x),ϕ(y)>

If the dot product is small, the functions are different; if it is large, there is more overlap. The Kernal trick then looks for transformations in the boundaries between the x and y by plotting the functions in multi-dimensional space in order to keep a linear classifier. Because we expect overlap in the nutrition, medication, lifestyle, symptoms, pain, and user defined metrics that predict whether or not an adverse symptom will occur, the Kernel trick will enable the combinations of factors to be plotted multi-dimensionally in order to define a natural linear divide between a symptom occurring vs. not occurring. This in turn will define which nutrition, medication, lifestyle, symptoms, pain, and user defined metrics and in which combination contribute to an adverse symptom outcome.

Random forest algorithms are a method for classification and regression that creates a series of decision trees to predict the alignment of a given input to a given tree. Specifically, random forests look at the predictive power of the full system of factors to determine the underlying function, plus noise. Random forest classification starts with a decision tree, wherein an input is entered at the top of the tree and travels down each branch. In the case of this application, the input would be an adverse symptom outcome, with each branch being the range of answers on a given predictive factor or series of predictive factors. Each day of inputted data would be its own tree, with the input being adverse symptom outcome and the branches for each of the predictors tracked. Random forests look at the average across a series of such trees to make a stronger prediction of an adverse outcome. The larger the number of trees, the more accurate the ultimate forest prediction. In this application, each tree is a day of data and the more days collected, the more accurate the predictions. Random forest algorithms identify the most important features. Random forests will therefore enable this application to identify the most salient factors from the tracked nutrition, medication, lifestyle, symptoms, pain, and user defined metrics. Random forests are also particularly adept at handling missing data, as is likely the case with user input daily logs. Random forest can help classify symptom groupings to better predict and manage symptoms. The method can include these steps: 1. Randomly select “K” features from total “m” features where k<<m. 2. Among the “K” features, calculate the node “d” using the best split point. 3. Split the node into daughter nodes using the best split. 4. Repeat the a to c steps until “I” number of nodes has been reached. 5. Build forest by repeating steps a to d for “n” number times to create “n” number of trees

Miscellaneous Classical Statistical Techniques 40 can include looking at distributions of data, means, mean comparisons, deviations, skewness, tracking over time, etc. These techniques are commonly used as a part of feature extraction (to supplement the user-submitted data when running the models).

Nearest neighbor algorithms can also be performed once a large enough group of users are using the application 10. A multi-dimensional nearest neighbor algorithm is used to find those individuals from existing sets, i.e. a K-Nearest Neighbor (KNN) algorithm. The KNN algorithm is a clustering algorithm and acts as a non-parametric untrained classifier that evaluates the overall similarity between two users based on the degree of differences across multiple features. The flexibility of such an algorithm allows consideration of many parameters when searching for pertinent context data. Weights on certain factors can vary depending on the type of symptom and food/nutrient. These similar user profiles are grouped into subsets to look for trends that can be used to optimize the suggestions for the user. While the KNN algorithm can be preferred, other clustering algorithms can also be used, such as, but not limited to, K-Means, Affinity Propagation, Mean Shift, Spectral Clustering, Support Vector Machines. One advantage of KNN over other techniques is that it is easily scalable across many dimensions. Further, from case-to-case the differing dimensions and weights are easily included.

The purpose of the KNN algorithm is to find users most similar to the present user. Once identified, the “neighboring” user data are used to evaluate the present user. To make the identification, we evaluate the differences in each parameter comprising the user data structure. While most commonly used with continuous values (weight, age, LDL level, etc.), the algorithm can be used with discrete values as well (race/ethnicity, familial history, presence of certain symptoms, DNA information etc.). The differences across each parameter are combined using a weighting scheme such that a normalized ‘distance’ is produced representing an overall difference metric between two users. The distance calculation between two users is achieved using a regression-type KNN algorithm. Key to the regression evaluations is the Mahalanobis distance. The Mahalanobis distance evaluates to a Euclidian distance since the covariance matrix is always the identity matrix, i.e., one parameter in this case is never to be compared independently with another parameter. The benefit of adapting the Mahalanobis distance instead of using pure Euclidian distance is that Mahalanobis distance includes the measurement of the number of deviations away from the norm. While the actual standard deviation is not always ideal, an equivalent term is used.

If the present user P₁ has a set of parameters where P₁={μ_{1P1}, μ_{2P1}, μ_{3P1}, . . . μ_{NP1}} and an arbitrary user, P_{β}, where P_{β}={μ_{1Pβ}, μ_{2Pβ}, μ_{3Pβ}, . . . μ_{NPβ}}, then the distance, D, between the two users is:

D₁(P₁,P_{β})={square root over (Σᵢ₌₁^{N}(μ_{iP1}−μ_{iPβ)}²)}

Several adaptations are needed to the above generalized equation. Mainly, handling a weighting schema. Most simply, a set of weights, W, should be created with each parameter in P being assigned a weight. Weights can be applied using any technique. Shown below is an intuitive 1-10 linear weighting schema. If W={ρ₁, ρ₂, ρ₃, . . . ρ_{N}}, then the distance, D, can be evaluated by:

D₂(P₁,P_{β})={square root over (Σᵢ₌₁^{N}ρᵢ(μ_{iP1}−μ_{iPβ)}²)}

In the above examples for D₁ and D₂ continuous values are used for μ_{N}. In this application, continuous values can be integers or rational numbers. Discrete values must be handled in a special manner. Since there is no intuitive value for the difference between two ethnicities, one must be manually supplied in a lookup table. Algorithmically, parameters with continuous values should be summated using the squared difference while parameters with continuous values are summated manually. The same W={ρ₁, ρ₂, ρ₃, . . . ρ_{N}} weighting schema applies to discrete parameters as well.

The threshold for evaluating whether or not another user is sufficiently similar to the present user is situational. The ideal number of similar subjects is to be optimized on a case-to-case basis when there exists sufficient training data.

KNN algorithms have been used before. For example, U.S. Pat. No. 10,123,748 (IBM) discloses a Patient Risk Analysis method that uses KNN to find similar patients. U.S. Pat. No. 7,730,063 discloses a personalized medicine method that also mentions KNN as a potential algorithm for finding similar patients. The present invention's ability to include continuous and discrete parameters as well as customized weights in the KNN differentiates over these prior art methods.

After the analysis, strongest trends 42, key performance indicators (KPIs) 44, and tracking over time 46 are sent to the output module 18 and displayed to the user. For example, predictor variables that meet a 60% or greater threshold are output to users with the output module 18 and flagged as potential causes of their symptoms or KPIs 44. Users are then encouraged to keep tracking to increase the predictive power. Predictor variables meeting a more stringent 90% threshold are flagged as likely causes, or strongest trends 42. Users are then encouraged to talk to their doctors to determine how they can improve their symptoms. Alternatively, the application 10 can be in communication with external databases and/or doctors/healthcare professionals that can suggests nutrition, medication, or lifestyle changes for the user to perform to improve their symptoms and to prevent triggers that have been identified. Users can review statistics of the outputs by week, month, or year with tracking over time 46.

The application 10 can also include any suitable alarms or notifications that can remind users to input data into the input module 12 or output variable module 14 at certain times of the day or daily. Such notifications can be pushed to the user's mobile devices such as a smart phone, smart watch, tablet, or desktop or laptop computer.

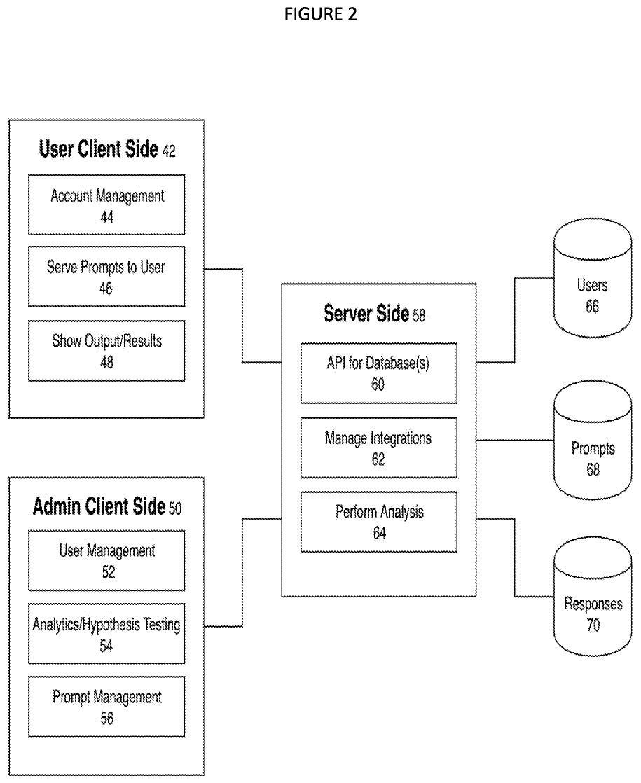

FIG. 2 shows a macro-level systems diagram. The User Client Side 42 includes the interactions the software has directly with the user. This includes interactions from native applications (iOS, Android), or web applications (accessed in a browser) and can include account management 44 (sign up, login, password management), serve prompts to user 46, and show output/results 48. The Admin Client Side 50 includes interactions “Admin” level users have access to, such as user management 52, analytics/hypothesis testing 54, and prompt management 56. The Server Side 58 outlines the major functions performed by the server. Application programming interface (API) for databases 60 can be performed. Integrations can be managed 62 including data from other health/nutrition trackers, fitness trackers, wearable devices, etc. Perform Analysis 64 refers to the breakdown represented in FIG. 1. Databases of users 66, prompts 68, and responses 70 can all be in electronic communication with the Server Side 58.

The present invention also provides for a method of tracking disease and mental health symptom triggers, by a user inputting data about nutrition, medication, lifestyle, symptoms, and user defined metrics in an application, performing an analysis on the data, and outputting a result from the data identifying daily activities that effect the user's disease/mental health and trigger symptoms, including strongest trends, key performance indicators, and tracking over time. This method can be performed with the application 10 as described above.

As mentioned above, the application 10 can integrate and analyze data (at 62) from outside devices 80 that measure physiological properties of the user and are preferably wearable medical devices. These outside devices 80 can include, but are not limited to, general fitness trackers (FitBits®, Apple® Watch), heartbeat trackers, heart rate trackers, skin temperature trackers, respiratory rate trackers, body posture trackers, eyesight trackers, blood oxygen trackers, glucose level trackers, sleep trackers, body temperature trackers, and skin conductance trackers. Any other suitable physiological data can also be collected. The outside devices 80 can be separate devices or a combination in a single device. Preferably, the outside devices 80 generally provide electrophysiological monitoring. Normally, one would go to a physician after a medical event (such as pain), their physiological conditions would be checked, and a wearable medical device would provide data to see what built up to the medical event to suggest activities not to do to avoid the medical event in the future. Using the application 10 with data from the outside devices 80 allows a user to discover triggers to their disease/mental health that do not necessarily correlate to a medical event that would not be found with just a physician examination and wearable device data alone.

Therefore, the present invention provides for a method of tracking disease and mental health symptom triggers, by a user inputting data about nutrition, medication, lifestyle, symptoms, and user defined metrics in an application, integrating a user's data from outside devices, performing an analysis on the data, and outputting a result from the data identifying daily activities that effect the user's disease/mental health and trigger symptoms, including strongest trends, key performance indicators, and tracking over time.

The application 10 can also integrate and analyze data from outside databases 90, especially having clinical trial data, such as clinics, electronic medical records (EMRs), pharmaceutical companies, private databases, or CROs, further described in U.S. Provisional Patent Application No. 62/878,066. Nearest neighbors can be identified as described above and related study or trial data can be identified in the outside databases 90 to be analyzed. By analyzing additional outside data from the outside databases 90, the application can find others who have similar data as the user and predict an adverse event or triggers to an adverse event. Further, the application 10 can integrate with weather monitoring systems. This is particularly relevant for migraines, as 75% of migraine sufferers report a correlation between weather and headaches (National Headache Foundation). Specifically, changes in humidity, temperature, and barometric pressure, as well as thunderstorms, and dry and dusty environments contribute to migraines. The application 10 can use these external weather data as a potential factor in predicting users' migraine incidents.

Therefore, the present invention provides for a method of tracking disease and mental health symptom triggers, by a user inputting data about nutrition, medication, lifestyle, symptoms, and user defined metrics in an application, integrating data from outside databases, performing an analysis on the data, and outputting a result from the data identifying daily activities that effect the user's disease/mental health and trigger symptoms, including strongest trends, key performance indicators, and tracking over time.

The application 10 can further combine the analysis of data from outside devices 80 with analysis of outside databases 90 in order to predict adverse events in a user. This allows for a user to know about the likelihood of an adverse event occurring at a later time point so that they can seek appropriate treatment (such as generally having surgery, having a heart bypass or cholesterol removed from arteries, or generally taking medicine) before the adverse event actually happens.

Therefore, the present invention provides for a method of preventing adverse events, by a user inputting data about nutrition, medication, lifestyle, symptoms, and user defined metrics as well as external data as described above in an application, integrating data from outside devices and outside databases, performing an analysis on the data, and outputting a result from the data identifying that an adverse event is likely to occur at a later time point. The method can further include the step of recommending that the user seek treatment for a condition that can cause the adverse event.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. An application for tracking disease, pain, and mental health symptom triggers stored on non-transitory computer readable media comprising:
an input module for inputting variables from a user in electronic communication with an output variable module;
an analysis module for analyzing input variables and output variables; and
an output module for presenting results to the user.

2. The application of claim 1, wherein the disease or disorder tracked is chosen from the group consisting of digestive disorders, migraines, anxiety attacks, and suicidal thoughts.

3. The application of claim 1, wherein the disease tracked is an infectious disease chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

4. The application of claim 1, wherein the pain tracked is from a source chosen from the group consisting of injury, surgery, cancer, fibromyalgia, arthritis, and peripheral neuropathy.

5. The application of claim 1, wherein said input module receives data from users in a nutrition question module, medication question module, and lifestyle question module.

6. The application of claim 1, wherein said output variable module includes a symptom question module, and user defined metrics question module.

7. The application of claim 1, wherein said input module receives data from outside devices chosen from the group consisting of general fitness trackers, heartbeat trackers, heart rate trackers, skin temperature trackers, respiratory rate trackers, body posture trackers, eyesight trackers, blood oxygen trackers, glucose level trackers, sleep trackers, body temperature trackers, skin conductance trackers, and combinations thereof.

8. The application of claim 1, wherein said input module receives data from outside databases chosen from the group consisting of clinics, electronic medical records (EMRs), pharmaceutical companies, private databases, weather monitoring systems, and CROs.

9. The application of claim 8, wherein said analysis module finds other individuals with similar data as the user to predict adverse events.

10. The application of claim 1, wherein said analysis module includes analysis methods of regressions, time series, random forest, classifiers, neural networks, support vector machines, AI/machine learning techniques, miscellaneous classical statistical techniques, and combinations thereof.

11. The application of claim 1, wherein said analysis module finds patterns between how users live and how they feel.

12. The application of claim 1, wherein said output module displays strongest trends, key performance indicators, and tracking over time and identifies disease, pain, and mental health triggers.

13. The application of claim 1, wherein said application is in electronic communication with external databases and healthcare professionals.

14. The application of claim 1, further including an alarm for reminding the user to input data into said input module and said output variable module.

15. A method of tracking disease, pain, and mental health triggers, including the steps of:
a user inputting data about nutrition, medication, lifestyle, symptoms, pain, and user defined metrics in an application stored on non-transitory computer readable media;
performing an analysis on the data; and
outputting a result from the data identifying daily activities that effect the user's disease/mental health/pain and trigger symptoms.

16. The method of claim 15, wherein said inputting step further includes the step of integrating a user's data from outside devices chosen from the group consisting of general fitness trackers, heartbeat trackers, heart rate trackers, skin temperature trackers, respiratory rate trackers, body posture trackers, eyesight trackers, blood oxygen trackers, glucose level trackers, sleep trackers, body temperature trackers, skin conductance trackers, and combinations thereof.

17. The method of claim 16, further including the step of discovering triggers that do not correlate to a medical event.

18. The method of claim 15, wherein said inputting step further includes the step of integrating data from outside databases chosen from the group consisting of clinics, electronic medical records (EMRs), pharmaceutical companies, private databases, weather monitoring systems, and CROs.

19. The method of claim 18, further including the step of predicting triggers based on individuals with similar data to the user.

20. The method of claim 15, wherein said performing an analysis step is further defined as performing an analysis method chosen from the group consisting of regressions, time series, random forest, classifiers, neural networks, support vector machines, AI/machine learning techniques, miscellaneous classical statistical techniques, and combinations thereof.

21. The method of claim 15, wherein the disease or disorder tracked is chosen from the group consisting of digestive disorders, migraines, anxiety attacks, and suicidal thoughts.

22. The method of claim 15, wherein the disease tracked is an infectious disease chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

23. The method of claim 15, wherein the pain tracked is from a source chosen from the group consisting of injury, surgery, cancer, fibromyalgia, arthritis, and peripheral neuropathy.

24. The method of claim 15, wherein said outputting step further includes displaying strongest trends, key performance indicators, and tracking over time.

25. The method of claim 15, further including the step of the user changing their lifestyle to prevent identified triggers.

26. A method of preventing adverse events, including the steps of:
a user inputting data about nutrition, medication, lifestyle, symptoms, pain and user defined metrics in an application stored on non-transitory computer readable media;
integrating data from outside devices and outside databases;
performing an analysis on the data; and
outputting a result from the data identifying that an adverse event is likely to occur at a later time point.

27. The method of claim 26, further including the step of recommending that the user seek treatment for a condition that can cause the adverse event.

28. The method of claim 26, wherein the adverse event is chosen from the group consisting of headaches, nausea, heart attacks, seizures, allergic reactions, hemorrhages, and tissue damage.

29. The method of claim 25, wherein said performing an analysis step further includes the step of predicting adverse events or triggers to an adverse event based on individuals with similar data to the user.
